(19) United States
(12) Plant Patent Application Publication
(10) Pub. No.: US 2002/0059669 P1
(43) Pub. Date: May 16, 2002

(54) LILY PLANT NAMED 'MIAMI'

(76) Inventor: CEES A. VAN DER VOORT, KATWIJK (NL)

Correspondence Address: FOLEY & LARDNER, WASHINGTON HARBOUR, 3000 K STREET, N.W., SUITE 500, WASHINGTON, DC, 20007-5109, US

(21) Appl. No.: 09/357,098

(22) Filed: Jul. 19, 1999

(51) Int. Cl. A01H 5/00

(52) U.S. Cl. PLT/315

(57) ABSTRACT

A new and distinct cultivar of Lily plant named ‘Miami’ characterized by having faster growing and faster flowering response than comparible varieties, large, erect flowers, and intense pink color with contrasting dark pink mid-vein.

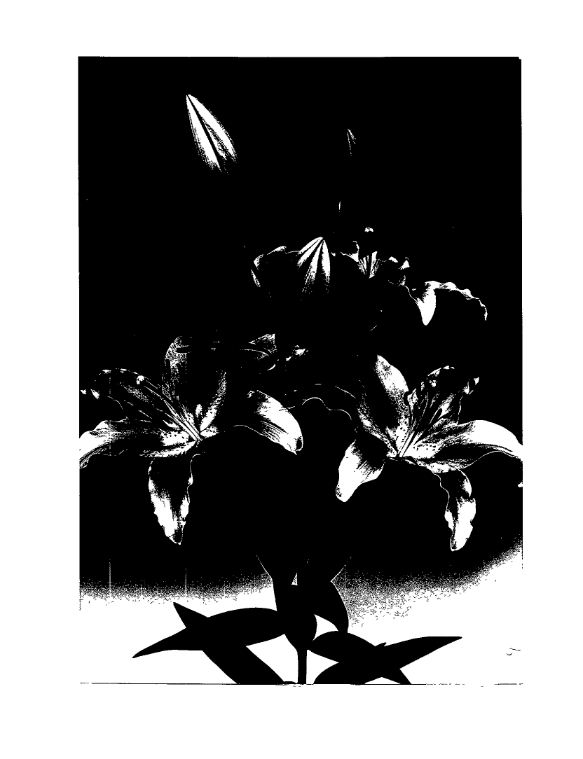

### BACKGROUND OF THE INVENTION

The present invention comprises a new and distinct cultivar of lily plant, botanically known as Lilium L., and hereinafter referred to by the cultivar name ‘Miami’.

‘Miami’ is a product of a planned breeding program which had the objective of creating new lily cultivars having faster flower response and a more intensive pink color.

‘Miami’ was originated from a hybridization made by the inventor in a controlled breeding program in Rijnsburg, the Netherlands in 1984. The male and female parents were unnamed seedlings of Lilium L. oriental hybrids. Both parents are proprietary cultivars used in the breeding program.

‘Miami’ was discovered and selected as one flowering plant within the progeny of the stated cross by the inventor, Cees A. van der Voort, on May 1, 1984 in a controlled environment in Rijnsburg, the Netherlands.

The first act of asexual reproduction of ‘Miami’ was accomplished when scales were taken from the initial selection in September 1987 in a controlled environment in Rijnsburg, the Netherlands by, or under the supervision of, Cees A. van der Voort. Horticultural examination of selected units initiated in May, 1987 has demonstrated that the combination of characteristics as herein disclosed for ‘Miami’ are firmly fixed and are retained through successive generations of asexual reproduction.

### BRIEF DESCRIPTION OF THE INVENTION

The following traits have been repeatedly observed and are determined to be basic characteristics of ‘Miami’ which in combination distinguish this lily as a new and distinct cultivar:

1. faster growing and faster flowering response than comparible varieties;

2. large, erect flowers; and

3. intense pink color with contrasting dark pink mid-vein.

‘Miami’ has not been observed under all possible environmental conditions. The phenotype of the new cultivar may vary significantly with variations in environment such as temperature, light intensity, and daylength without any change in genotype. The following observations, measurements, and comparisons describe plants grown in greenhouse/field conditions in Rijnsburg, the Netherlands under conditions which approximate those generally used in commercial practice.

Of the many commercial cultivars known to the present inventor, the most similar in comparison to ‘Miami’ is the cultivar ‘Berlin’. In comparison to ‘Berlin’, ‘Miami’ has a significantly faster flowering response.

### BRIEF DESCRIPTION OF THE DRAWING

The accompanying color photographic drawing shows typical plant, bulb and flower characteristics of ‘Miami’, with colors being as true as possible with illustrations of this type.

### DETAILED BOTANICAL DESCRIPTION

In the following description, color references are made to The Royal Horticultural Society Colour Chart (RHS). The color values were determined between 10 and 12 o'clock on Jun. 10, 1992 at Rinjsburg, the Netherlands.

Origin: Selected crossing from a controlled hybridization program

Parentage:

Female parent.—Unnamed seedling.

Male parent.—Unnamed seedling.

Classification:

Botanical.—A hybrid of the genus Lilium L.

Commercial.—Oriental Lily cv. ‘Miami’.

Plant:

Form.—A single stem carrying numerous, alternately arranged petals.

Height.—Medium for the mature plant, depending on cultivation conditions.

Growth Habit.—Vigorous.

Stem.—Circular in transverse section; anthocyanin pigmentation present in spots and stripes; internodal length uniform.

Foliage:

Quantity.—Small to medium, depending on cultivation conditions.

Size of leaf.—Mature leaves are medium in length and medium in width.

Shape of leaf.—Generally ovate.

Texture.—Hardly any pubescence

Color.—Medium green, RHS 135A.

Bulb:

Size.—16 to 18 cm in circumference.

Color.—White.

Bud:

Form.—Tapering like a lancet.

Size.—11 cm in length, depending on cultivation conditions.

Opening.—Unfolds normally.

Color.—RHS 65A.

Tepals.—Folded, three visible.

Flower:

Blooming habit.—Raceme.

Size.—Short to medium, depending on cultivation conditions.

Borne.—Upright to horizontal.

Shape.—Generally, hexegonal star in form and bowl-shaped in cross-section.

Tepalage:

Number of Tepals.—6.

Arrangement.—Three inner and three outer.

Color:

Upper Surface.—Pink, RHS 65A to RHS 62B.

Lower Surface.—Light pink, RHS 65B.

Spotting or Marking.—Pink red spots, the color of the mid-vein is yellow-green variegated to yellow.

Pedicel:

Length.—Approximately 11.5 cm (first flower), depending on cultivation conditions.

Color.—Pink.

Form.—Strongly curved; medium to strong reflex.

Texture.—Papillose.

Disease resistance:

Fragrance.—Present.

Lasting Quality.—Excellent.

Reproductive organs:

Stamens and anthers:

Arrangements.—Anthers form a generally circular pattern at their upper ends.

Number.—6.

Length:

Filaments Color.—White.

Pollen color.—Orange brown.

Anther color.—Red brown.

Pistils.—Green.

Stigma.—Grey.

Ovaries.—Trianglar in shape.

I claim:

1. A new and distinct cultivar of Lily plant named ‘Miami’, as illustrated and described herein.
